(19) United States
(12) Patent Application Publication
(10) Pub. No.: US 2020/0297203 A1
(43) Pub. Date: Sep. 24, 2020

(54) ENDOSCOPE

(71) Applicant: OLYMPUS CORPORATION, Tokyo (JP)

(72) Inventor: Takayoshi TOGINO, Tokyo (JP)

(73) Assignee: OLYMPUS CORPORATION, Tokyo (JP)

(21) Appl. No.: 16/894,475

(22) Filed: Jun. 5, 2020

Related U.S. Application Data
(63) Continuation of application No. PCT/JP2017/043868, filed on Dec. 6, 2017.

(51) Int. Cl. A61B 1/05; A61B 1/307; A61B 1/00; A61B 1/07

(52) U.S. Cl. CPC A61B 1/05; A61B 1/307; A61B 1/00013; A61B 1/07; A61B 1/00009; A61B 1/00071

(57) ABSTRACT

An endoscope includes a circular cylindrical member, a distal end member, and an image forming optical system. The circular cylindrical member has an inner peripheral surface and an outer peripheral surface. A space between the inner peripheral surface and the outer peripheral surface is filled with a transparent material. The distal end member is positioned at one end of the circular cylindrical member. The image forming optical system is disposed at an interior of the circular cylindrical member. The image forming optical system includes only transmitting surfaces and all the transmitting surfaces are disposed such that a normal of a plane at a point intersecting with the optical axis is aligned with the optical axis. The image forming optical system has a curvature of field, and the following conditional expression (1) is satisfied:

−10<P′<−0.8 (1).

### CROSS-REFERENCE TO RELATED APPLICATION

The present application is a continuation application of International Application No. PCT/JP2017/043868 filed on Dec. 6, 2017, the entire contents of which are incorporated herein by reference.

### BACKGROUND

### Technical Field

The present disclosure relates to an endoscope, and relates to an endoscope which enables observation of a pipe and observation of internal organs of a urinary system for example.

### Description of the Related Art

For instance, in an observation of a water pipe and an observation of a pipe of a steam generator, the observation is carried out through water. In an observation of an inside of a fuel tank, the observation is carried out through oil. In a case in which a type of oil is light diesel oil for example, the observation is to be carried out through a liquid having a refractive index of 1.45.

Moreover, in an observation of the internal organs of the urinary system, the observation is carried out through urine. In an observation of internal organs of a digestive system and an observation of joints, a physiological salt solution being used as a reflux liquid, the observation is carried out through the physiological salt solution.

In an observation of an object located in a space filled with a liquid (hereinafter, ‘in-liquid observation’), a wide field of view is sought. Therefore, it is desirable that an angle of view of an optical system be wide. Optical systems having a wide angle of view are disclosed below.

An optical system of Japanese Patent No. 4544939 Publication has a rotationally symmetric transparent medium. The transparent medium has at least two internal reflecting surfaces and at least two refractive surfaces. Moreover, the transparent medium is either mounted on an incidence side of an image forming lens having a positive refractive power, or mounted on an emergence side of a projection lens having a positive refractive power.

An optical system of Japanese Patent No. 5025354 Publication has an optical element consisting of a transparent medium, a front unit, an aperture stop, and a rear unit. The transparent medium has a first transmitting surface, a first reflective surface, a second reflective surface, and a second transmitting surface.

An optical system of Japanese Patent No. 5753326 Publication and an optical system of Japanese Patent No. 6064105 Publication have a front unit having a negative refractive power, an aperture stop, and a rear unit having a positive refractive power.

An optical system of Japanese Patent No. 5214161 Publication has a rotationally symmetric front unit, and a rotationally symmetric rear unit having a positive refractive power. The front unit has two transmitting surfaces. Moreover, a transparent circular cylindrical body is disposed around the optical system.

A unit which includes a transparent member is disclosed in Japanese Patent No. 3790866 Publication. In Japanese Patent No. 3790866 Publication, a cap portion is disposed around an endoscope distal end.

When an optical system envisaged for an observation of an object located in a space filled with air (hereinafter, referred to as ‘in-air observation’) is used for an optical system used for the in-liquid observation, since a refractive index of an object space changes from a refractive index of air to a refractive index of a liquid, it is difficult to achieve a wide field of view.

In the optical system disclosed in the Japanese Patent No. 4544939 Publication and the optical system disclosed in Japanese Patent No. 5025354 Publication, it is possible to carry out an observation in a direction orthogonal to an optical axis (hereinafter, referred to as ‘side view direction’). These optical systems are optical systems to be used for in-air observation.

Moreover, in these optical systems, a reflective surface is used in the optical systems.

The optical system disclosed in Japanese Patent No. 5753326 Publication and the optical system disclosed in Japanese Patent No. 6064105 Publication are optical systems used for in-water observation. In these optical systems, an angle of view in a direction along an optical axis (hereinafter, referred to as ‘direct view direction’) has been widened.

In the optical system disclosed in Japanese Patent No. 5214161 Publication, it is possible to carryout an observation in the side view direction. This optical system is an optical system envisaged for observing an object by bringing the object in close contact with an outer circular cylindrical surface of the transparent circular cylindrical body.

The cap portion disclosed in Japanese Patent No. 3790866 Publication is a portion to be used in the in-air observation.

### SUMMARY

An endoscope according to at least some embodiments of the present disclosure includes:

a circular cylindrical member,

a distal end member, and

an image forming optical system,

wherein:

the circular cylindrical member has an inner peripheral surface and an outer peripheral surface,

a space between the inner peripheral surface and the outer peripheral surface is filled with a transparent material having a refractive index higher than 1,

the distal end member is positioned at one end of the circular cylindrical member,

the image forming optical system is disposed at an interior of the circular cylindrical member such that an optical axis of the image forming optical system and a central axis of the circular cylindrical member are aligned or become parallel,

due to the image forming optical system, an object plane positioned at an outer side of the outer peripheral surface and an image plane of the image forming optical system become conjugate,

the image forming optical system includes only transmitting surfaces,

all the transmitting surfaces are disposed such that a normal of a plane at a point intersecting with the optical axis is aligned with the optical axis,

the image forming optical system has a curvature of field, and

the following conditional expression (1) is satisfied:

−10<P′<−0.8 (1),

where,

P′ denotes Petzval sum, and is expressed by the following expression,

$${P}^{′} = {n}^{′} {\sum }_{i = 1}^{k} \frac{1}{{r}_{i}} ( \frac{1}{{n}_{i}^{′}} - \frac{1}{n} ) ,$$

rᵢ denotes a radius of curvature of an i^{th} transmitting surface,

n′ᵢ denotes a refractive index at an emergence side of the i^{th} transmitting surface,

nᵢ denotes a refractive index at an incidence side of the i^{th} transmitting surface,

n′ denotes a refractive index of an image space,

i denotes a number of a transmitting surface, and

k denotes the total number of transmitting surfaces.

### BRIEF DESCRIPTION OF THE DRAWINGS

FIG. 1 is a diagram showing an optical unit of an endoscope of the present embodiment;

FIG. 2A and FIG. 2B are diagrams showing an image forming relationship of an optical system having a curvature of field;

FIG. 3 is a diagram showing an appearance of a light beam refracted by a circular cylindrical member;

FIG. 4 is a diagram showing an appearance of a light beam at a meridional cross section;

FIG. 5 is a diagram showing an appearance of a light beam at a sagittal cross section;

FIG. 6 is a graph showing a relationship between a predetermined difference in positions and a predetermined angle;

FIG. 7A and FIG. 7B are diagrams showing an appearance of refraction of a light beam at the sagittal cross section;

FIG. 8 is a diagram showing an appearance of a light beam at the meridional cross section;

FIG. 9 is a diagram showing another optical unit of the present embodiment;

FIG. 10 is a lens cross-sectional view of an image forming optical system of an example 1;

FIG. 11 is a lens cross-sectional view of an image forming optical system of an example 2;

FIG. 12 is a lens cross-sectional view of an image forming optical system of an example 3;

FIG. 13 is a lens cross-sectional view of an image forming optical system of an example 4;

FIG. 14A, FIG. 14B, and FIG. 14C are aberration diagrams of the image forming optical system of the example 1;

FIG. 15A, FIG. 15B, and FIG. 15C are aberration diagrams of the image forming optical system of the example 2;

FIG. 16A, FIG. 16B, and FIG. 16C are aberration diagrams of the image forming optical system of the example 3;

FIG. 17A, FIG. 17B, and FIG. 17C are aberration diagrams of the image forming optical system of the example 4;

FIG. 18 is a diagram showing a first example of an optical unit;

FIG. 19 is a diagram showing a second example of the optical unit;

FIG. 20 is a diagram showing a first example of an insertion portion of the present embodiment;

FIG. 21 is a diagram showing a second example of the insertion portion of the present embodiment;

FIG. 22 is a diagram showing a third example of the insertion portion of the present embodiment;

FIG. 23 is a diagram showing an arrangement example of an illuminating optical system; and

FIG. 24A and FIG. 24B are diagrams showing examples of an endoscope of the present embodiment.

### DETAILED DESCRIPTION

Prior to the explanation of examples, action and effect of embodiments according to certain aspects of the present disclosure will be described below. In the explanation of the action and effect of the embodiments concretely, the explanation will be made by citing concrete examples. However, similar to a case of the examples to be described later, aspects exemplified thereof are only some of the aspects included in the present disclosure, and there exists a large number of variations in these aspects. Consequently, the present disclosure is not restricted to the aspects that will be exemplified.

An endoscope of the present embodiment includes a circular cylindrical member, a distal end member, and an image forming optical system. The circular cylindrical member has an inner peripheral surface and an outer peripheral surface, a space between the inner peripheral surface and the outer peripheral surface is filled with a transparent material having a refractive index higher than 1, the distal end member is positioned at one end of the circular cylindrical member, the image forming optical system is disposed at an interior of the circular cylindrical member in order that an optical axis of the image forming optical system and a central axis of the circular cylindrical member are aligned or become parallel, due to the image forming optical system, an object plane positioned at an outer side of the outer peripheral surface and an image plane of the image forming optical system become conjugate, the image forming optical system includes only transmitting surfaces, all the transmitting surfaces are disposed in order that a normal of a plane at a point intersecting with the optical axis is aligned with the optical axis, the image forming optical system has a curvature of field, and the following conditional expression (1) is satisfied:

−10<P′<−0.8 (1),

where,

P′ denotes Petzval sum, and is expressed by the following expression,

$${P}^{′} = {n}^{′} {\sum }_{i = 1}^{k} \frac{1}{{r}_{i}} ( \frac{1}{{n}_{i}^{′}} - \frac{1}{n} ) ,$$

rᵢ denotes a radius of curvature of an i^{th} transmitting surface,

n′ᵢ denotes a refractive index at an emergence side of the i^{th} transmitting surface,

nᵢ denotes a refractive index at an incidence side of the i^{th} transmitting surface,

n′ denotes a refractive index of an image space,

i denotes a number of a transmitting surface, and

k denotes the total number of transmitting surfaces.

The endoscope of the present embodiment includes the circular cylindrical member, the distal end member, and the image forming optical system. The circular cylindrical member can It is possible to form an optical unit with the circular cylindrical member, the distal end member, and the image forming optical system. The optical unit is disposed at a distal end of an insertion portion of the endoscope. The description will be made below in order of the optical unit and the insertion portion.

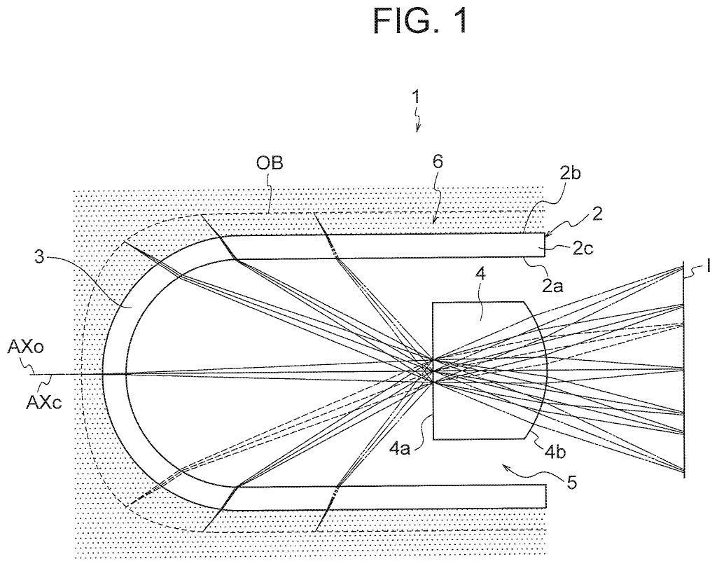

The optical unit of the endoscope of the present embodiment (hereinafter, referred to as ‘optical unit of the present embodiment’) is shown in FIG. 1. An optical unit 1 includes a circular cylindrical member 2, a distal end member 3, and an image forming optical system 4. The circular cylindrical member 2 has an inner peripheral surface 2a and an outer peripheral surface 2b. A space between the inner peripheral surface 2a and the outer peripheral surface 2b is filled with a transparent material 2c having a refractive index higher than 1.

The distal end member 3 is positioned at one end of the circular cylindrical member 2. The distal end member 3 is formed by a transparent medium. A shape of the distal end member 3 is substantially semispherical, but it is not restricted to the semispherical shape. It may be flat for example. Moreover, as it will be described later, the distal end member 3 may be formed by an opaque medium.

A holding member (not shown in the diagram) is disposed at the other end of the circular cylindrical member 2. A hermetically sealed space is formed by the circular cylindrical member 2, the distal end member 3, and the holding member. It is possible to dispose the image forming optical system 4 in this hermetically sealed space. Airtightness being maintained at an inner side of the circular cylindrical member 2, it is possible to position the image forming optical system 4 in air, and to protect from dirt and the like. It is possible to dispose an illuminating optical system (not shown in the diagram) in the hermetically sealed space.

In the optical unit 1, the circular cylindrical member 2 and the distal end member 3 are made of separate members. The circular cylindrical member 2 and the distal end member 3 may be integrated by sticking for example. The circular cylindrical member 2 and the distal end member 3 may be made of one member.

The image forming optical system 4 is disposed at an interior of the circular cylindrical member 2. At this time, an optical axis AXo of the image forming optical system 4 and a central axis AXc of the circular cylindrical member 2 may be or may not be aligned. In a case in which the two axes are not aligned, an arrangement is to be made so that the two axes become parallel. In FIG. 1, the image forming optical system 4 is disposed at the interior of the circular cylindrical member 2 such that the optical axis AXo and the central axis AXc are aligned.

An object plane OB and an image plane I are conjugate due to the image forming optical system 4. In FIG. 1, the object plane OB is indicated by a dashed line. The object plane OB is positioned at an outer side of the outer peripheral surface 2b. The object plane OB is illuminated by the illuminating optical system (not shown in the diagram).

The image forming optical system 4 is formed by one single lens. The image forming optical system 4 has a transmitting surface 4a and a transmitting surface 4b. In such manner, the image forming optical system 4 is formed by only transmitting surfaces. In the image forming optical system 4, an aperture stop is located on the transmitting surface 4a.

The transmitting surface 4a and the transmitting surface 4b are disposed such that a normal of a plane at a point intersecting with the optical axis AXo is aligned with the optical axis AXo. In such manner, all the transmitting surfaces of the image forming optical system 4 are disposed such that the normal of the plane at the point intersecting with the optical axis AXo is aligned with the optical axis AXo.

The circular cylindrical member 2 is positioned in a side view direction. The distal end member 3 is positioned in a direct view direction. The circular cylindrical member 2 and the distal end member 3 are formed of a transparent material. Therefore, in the optical unit 1, an image in the side view direction is formed on the image plan I via the circular cylindrical member 2, and an image in a direct view direction is formed on the image plane I via the distal end member 3.

It is possible to dispose an image sensor on the image plane I, for example. In this case, an optical image of an object formed on the image plane I is electronically converted by the image sensor. Accordingly, it is possible to acquire an image of the object. The acquired image is transmitted to an image processing apparatus by a transmitting unit, for example. As mentioned above, the airtightness being maintained at the inner side of the circular cylindrical member 2, it is possible to position the image forming optical system 4 and the image sensor in air, and to protect from dirt and the like.

A first space 5 is a space formed by a space positioned at the inner side of the circular cylindrical member 2 and a space positioned at an inner side of the distal end member 3. The first space 5 is filled with air. A second space 6 is a space formed by a space positioned at an outer side of the circular cylindrical member 2 and a space positioned at an outer side of the distal end member 3. The second space 6 is filled with a liquid.

The object plane OB is positioned in the second space 6. An image of the object plane OB is formed in the first space 5. Accordingly, the second space 6 corresponds to the object space, and the first space 5 corresponds to the image space. The object plane OB being located in the second space 6, an image of the object plane OB is formed via the liquid.

The object plane OB in the side view direction is a circular cylindrical surface. Whereas, the image plane I is a flat surface. Accordingly, in the image forming optical system 4, an image of the circular cylindrical surface has to be formed on a flat surface.

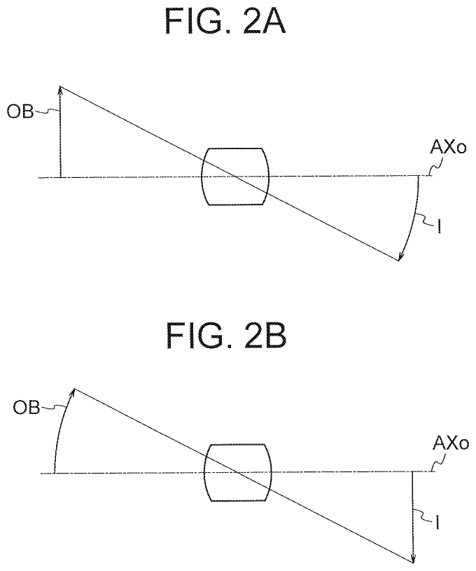

FIG. 2A and FIG. 2B are diagrams showing an image forming relationship of an optical system having a curvature of field. FIG. 2A indicates a case in which an object plane is a flat surface, and FIG. 2B shows a case in which the object plane is a curved surface.

A sign of Petzval sum indicates a direction of occurrence of the curvature of field, and a value of Petzval sum indicates an amount of occurrence of the curvature of field. Generally, in an optical system having a positive refractive power, the sign of Petzval sum becomes minus. In an optical system in which the sign of Petzval sum is minus, as shown in FIG. 2A, in the case in which the object plane OB is a flat surface, a curved surface with a concave surface directed toward an object side is formed on the image plane I.

In an optical system, it is possible to reverse an object and an image. Therefore, when the object plane OB in FIG. 2A is deemed as an image plane, and the image plane I is deemed as an object plane, the object plane OB becomes a curved surface with a concave surface directed toward an image side as shown in FIG. 2B. Whereas, the image plane I becomes a flat surface. In such manner, in an optical system in which the sign of Petzval sum is minus, it is possible to form an object of a curved surface on a flat surface. When the object plane OB is a curved surface with a concave surface directed toward the image side, a focused range becomes wide. Therefore, it is preferable to use the optical system in which the sign of Petzval sum is minus.

In the optical unit of the present embodiment, the optical system in which the sign of the Petzval sum is minus is used for the image forming optical system 4. In this case, since the image forming optical system 4 has the curvature of field, even when the object plane is a curved surface, it is possible to form an image of the object plane on a flat surface.

For forming a sharp optical image of an object over even wider range, it is desirable to generate the curvature of field of an appropriate amount in the image forming optical system. In other words, it is desirable to make the value of Petzval sum appropriate.

The formation of an optical image of the object is carried out via the circular cylindrical member 2. Both the inner peripheral surface 2a and the outer peripheral surface 2b are cylindrical surfaces. In other words, the inner peripheral surface 2a and the outer peripheral surface 2b don't have a refractive power in a direction along the optical axis AXo, but have a refractive power in a direction orthogonal to the optical axis AXo. Therefore, an astigmatism occurs when a light ray passes through the inner peripheral surface 2a and through the outer peripheral surface 2b. When the astigmatism occurs largely, formation of the sharp optical image of the object becomes difficult.

The first space 5 being filled with air, a refractive index n5 in the first space 5 is 1.0. Here, a refractive index n2c of the material 2c is assumed to be 1.51, and a refractive index n6 in the second space 6 is assumed to be 1.33. In this case, a magnitude correlation of refractive indices becomes as follows.

n5<n2c

n2c>n6

From the first space 5 toward the second space 6, the refractive index varies in order of n5, n2c, n6. A difference in refractive index in this direction becomes as follows.

n5−n2c<0

n2c−n6>0

A difference in refractive index on both sides of the inner peripheral surface 2a becomes a minus value, and a difference in refractive index on both sides of the outer peripheral surface 2b becomes a plus value. Consequently, a direction in which the astigmatism occurs at the inner peripheral surface 2a becomes opposite to a direction in which the astigmatism occurs at the outer peripheral surface 2b.

Moreover, since n5<n6, the magnitude correlation of a difference in refractive index becomes as follows.

|n5−n2c|>|n2c−n6|

As the difference in refractive index becomes longer, the astigmatism becomes larger. Therefore, the astigmatism that occurs at the inner peripheral surface 2a becomes larger than the astigmatism that occurs at the outer peripheral surface 2b.

In a case in which an outer side of the circular cylindrical member is a liquid, when the circular cylindrical member 2 having a small diameter is used, a large astigmatism occurs particularly at the inner peripheral surface 2a. Consequently, formation of a sharp image becomes difficult.

In such manner, an effect on the formation of an image is larger for the astigmatism that occurs at the inner peripheral surface 2a as compared to the astigmatism that occurs at the outer peripheral surface 2b. The astigmatism that occurs at the inner peripheral surface 2a will be described below.

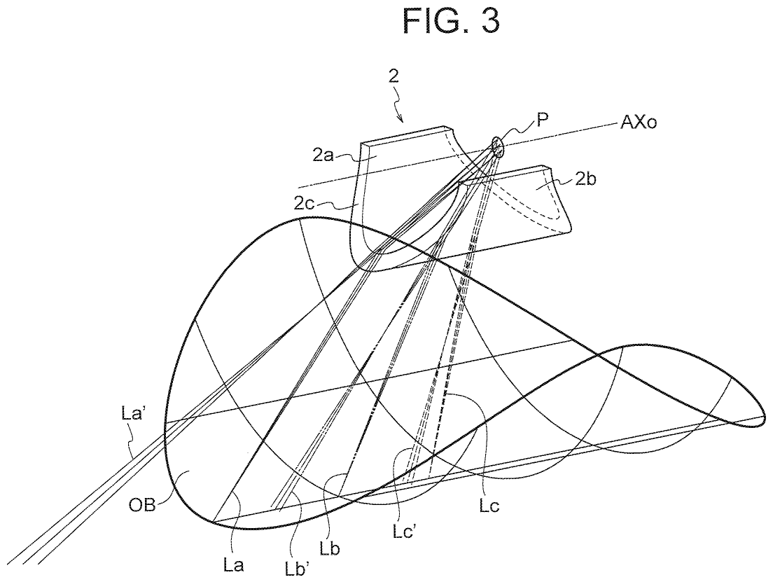

FIG. 3 is a diagram showing an appearance of a light beam refracted by a circular cylindrical member. An optical axis of an image forming optical system is aligned with a central axis of the circular cylindrical member. However, the image forming optical system is not shown in the diagram. Instead, an entrance pupil P of the image forming optical system is shown in the diagram. In FIG. 3, an appearance of a light beam from the entrance pupil P up to the object plane OB is shown. Only a part of the object plane OB and a part of the circular cylindrical member 2 is depicted.

Values related to the circular cylindrical member 2, the entrance pupil P, and the object plane OB are as follows.

inner peripheral surface 2a: circular cylindrical surface of 1.0 mm diameter

outer peripheral surface 2b: circular cylindrical surface of 1.2 mm diameter

refractive index of the material 2c: 1.5163

diameter of the entrance pupil P: 0.1 mm

object plane OB: circular cylindrical surface of 4 mm diameter

As a distance from the optical system to an object point becomes longer, the astigmatism becomes larger. As shown in FIG. 2A, in a case in which the object plane OB is a flat surface perpendicular to the optical axis AXo, a distance from an object point on the object plane OB to the optical system becomes longer as the object point recedes from the optical axis AXo. Therefore, when formation of an image of the object plane OB is carried out via the circular cylindrical member 2, the astigmatism which occurs at the inner peripheral surface 2a becomes larger as the object point on the object plane OB recedes from the optical axis AXo.

Whereas, as shown in FIG. 2B, in a case in which the object plane OB is a curved surface with a concave surface directed toward image side, a distance from the object point on the object plane OB to the optical system becomes shorter as the object point recedes from the optical axis AXo. Therefore, when formation of an image of the object plane OB is carried out via the circular cylindrical member 2, the astigmatism which occurs at the inner peripheral surface 2a becomes smaller as the object point on the object plane OB recedes from the optical axis.

When a position of the object point is brought closer to the optical system, a refraction effect of the optical system becomes smaller and smaller. Therefore, as shown in FIG. 3, the object plane OB is made a semispherical concave surface from the flat surface, and furthermore, the object plane OB is brought closer to the inner peripheral surface 2a. By making such arrangement, it is possible to suppress the occurrence of astigmatism at the inner peripheral surface 2a.

A relation between a distance from the inner peripheral surface 2a to the object plane and the astigmatism will be described below. In FIG. 3, light beams La, Lb, and Lc of a real image and light beams La′, Lb′, and Lc′ of a virtual image are depicted. The light beams of the real image are light beams incident on the entrance pupil P from the object plane OB. The light beams of the virtual image are light beams in which light beams of the real image from the inner peripheral surface 2a to the entrance pupil P are extended toward the object plane OB side.

For the light beams La, Lb, and Lc, an angle made by a principal light ray and the optical axis AXo differs for each light beam. The angle made by the principal light ray and the optical axis becomes larger for the light beams in order of the light beam La, the light beam Lb, and the light beam Lc.

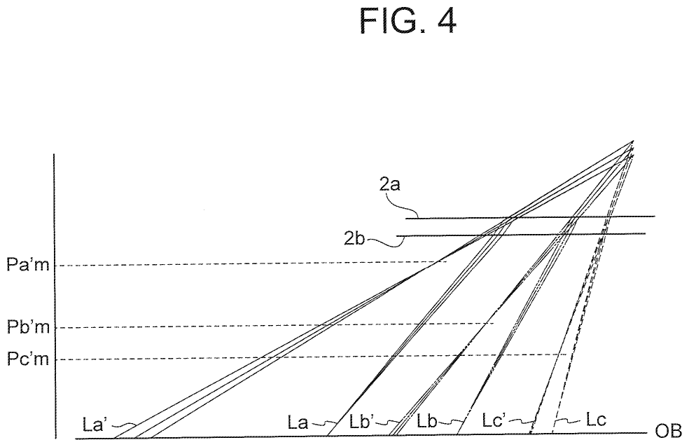

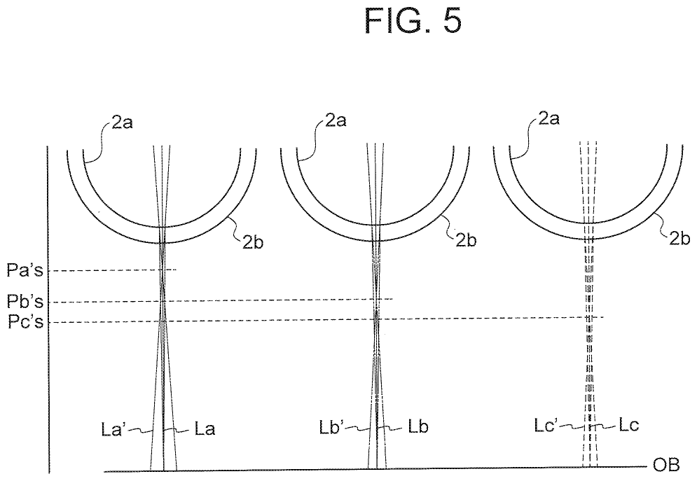

Each of the light beams La, Lb, and Lc includes a light beam at a meridional cross section and a light beam at a sagittal cross-section. FIG. 4 is a diagram showing an appearance of a light beam at the meridional cross section. FIG. 5 is a diagram showing an appearance of a light beam at a sagittal cross section.

At the meridional cross section, as shown in FIG. 4, for the light beam La′, a diameter of the light beam becomes the smallest at a position Pa′m; for the light beam Lb′, a diameter of the light beam becomes the smallest at a position Pb′m; and for the light beam Lc′, a diameter of the light beam becomes the smallest at a position Pc′m. The positions at which the light beams become the smallest (hereinafter, referred to as ‘positions of the smallest diameter’) are side-by-side in order of the position Pa′m, the position Pb′m, and the position Pc′m, closer from the inner peripheral surface 2a.

At the sagittal cross section, as shown in FIG. 5, for the light beam La′, the diameter of the light beam becomes the smallest at a point Pa's; for the light beam Lb′, the diameter of the light beam becomes the smallest at a position Pb's; and for the light beam Lc′, the diameter of the light becomes the smallest at a position Pc's. The positions of the smallest diameter are side-by-side in order of the position Pa's, the position Pb's, and the position Pc's, closer from the inner peripheral surface 2a.

At the meridional cross section, the inner peripheral surface 2a does not have a refractive power. Whereas, at the sagittal cross section, the inner peripheral surface 2a has a refractive power. Therefore, the position Pa′m and the position Pa's do not coincide. Similarly, the position Pb′m and the position Pb's do not coincide. Moreover, the position Pc′m and the position Pc's do not coincide.

A difference in the two positions is generated by a difference in the refractive power at the meridional cross section and the refractive power at the sagittal cross section. The difference in the refractive power at the meridional cross section and the refractive power at the sagittal cross section is one of the factors of occurrence of the astigmatism. Therefore, by using a predetermined difference in positions, it is possible to carry out evaluation of the astigmatism.

The predetermined difference in positions is a difference of the position of the smallest diameter at the meridional cross section and the position of the smallest diameter at the sagittal cross section. From FIG. 4 and FIG. 5, the predetermined difference in positions becomes as follows.

difference in the position Pa′m and the position Pa's

difference in the position Pb′m and the position Pb's

difference in the position Pc′m and the position Pc's

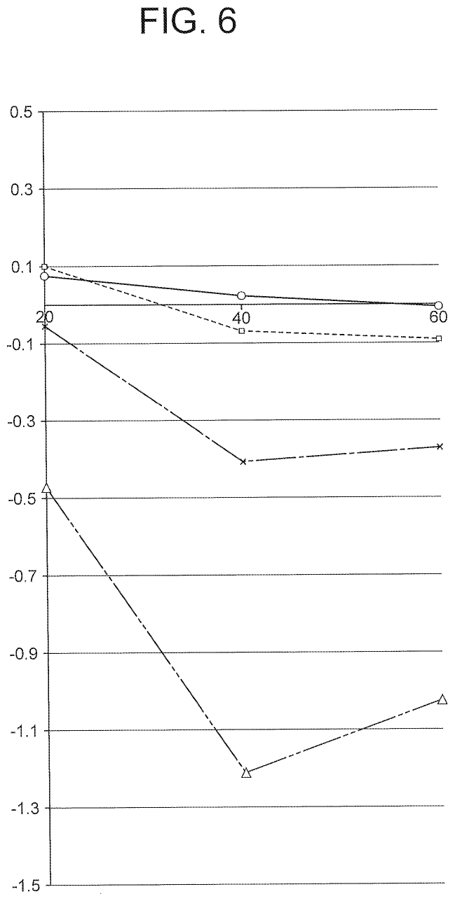

FIG. 6 is a graph showing a relationship between the predetermined difference in positions and a predetermined angle. A vertical axis shows the predetermined difference in positions and a horizontal axis shows the predetermined angle. The predetermined angle is an angle made by a central axis and a principal light ray at an outer side of the circular cylindrical member.

The graph in FIG. 6 indicates a result in which a simulation is carried out. Conditions for the simulation are as follows.

inner peripheral surface: circular cylindrical surface of which a diameter is 0.6 mm.

outer peripheral surface: circular cylindrical surface of which a diameter is 1 mm.

refractive index between the inner peripheral surface and the outer peripheral surface: 1.516.

refractive index between the inner peripheral surface and the image forming optical system: 1.

refractive index between the outer peripheral surface and the object plane: 1.33.

The inner side of the circular cylindrical member is filled with air and the outer side of the circular cylindrical member is filled with water. Moreover, the optical axis of the image forming optical system and the central axis of the circular cylindrical member are aligned. Therefore, the predetermined angle can be deemed as an angle of view of the image forming optical system. The position of the smallest diameter is represented with reference to the optical axis of the image forming optical system. A position at which the spot diagram becomes the smallest is assumed to be the position of the smallest diameter.

In the simulation, an object distance in the side view direction is caused to differ, and the predetermined difference in positions is calculated. The object distance in the side view direction is a distance from the optical axis to the object plane, on a plane orthogonal to the optical axis of the image forming optical system.

A relationship between a type of lines in the graph and the object distance in the side view direction is as follows. As mentioned above, the outer peripheral surface is a circular cylindrical surface of which a diameter is 1 mm. Therefore, in a case in which the object distance in the side view direction is 0.5 mm, the object surface is aligned with the outer peripheral surface.

| | Object distance in | |
|---|---|---|
| Type of line | side view direction | |
| solid line | 0.5 | mm |
| dashed line | 1 | mm |
| alternate long and short dashed line | 2 | mm |
| alternate long and two short dashes line | 4 | mm |

A relationship between the object distance in the side view direction and the predetermined difference in positions is as follows. As the object distance in the side view direction becomes larger, the predetermined difference in positions becomes larger. This signifies that, as the object distance in the side view direction becomes larger, an amount of occurrence of the astigmatism becomes larger. Moreover, the astigmatism occurs in a minus direction.

| | |
|---|---|
| Object distance in | Predetermined difference in positions |

| | | |
|---|---|---|
| side view direction | minimum | maximum |

| | | | |
|---|---|---|---|
| 0.5 | mm | 0 | 0.08 |
| 1 | mm | −0.1 | 0.1 |
| 2 | mm | −0.4 | −0.05 |
| 4 | mm | −1.21 | −0.47 |

In a case in which the object distance in the side view direction is 0.5 mm, an amount of occurrence of astigmatism is the smallest. Therefore, in the optical unit of the present embodiment, it is desirable to form an image of the object plane in a state of the object plane and the outer peripheral surface aligned.

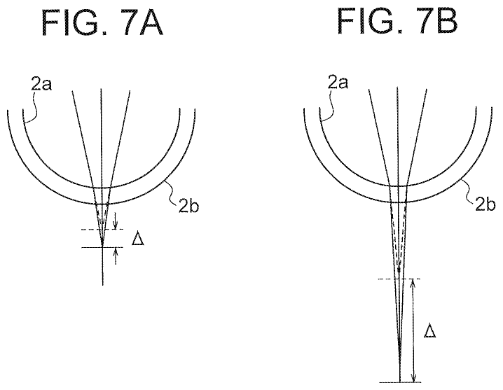

FIG. 7A and FIG. 7B are diagrams showing an appearance of refraction of a light beam at the sagittal cross section. FIG. 7A shows a case in which the object distance in the side view direction is short, and FIG. 7B shows a case in which the object distance in the side view direction is long. When a light beam passes through the inner peripheral surface 2a, at the sagittal cross-section, the light beam is spread due to refraction. Solid lines show a light beam refracted, and dashed lines show a light beam not refracted.

In a case in which the object distance in the side view direction is short, or in other words, in a case in which the position of the object plane is near the outer peripheral surface, spreading of the light beam is small. In a case in which the object distance in the side view direction is long, or in other words, in a case in which the position of the object plane is far from the outer peripheral surface, the spreading of the light beam is small. Therefore, a difference 4 in positions between a light collecting position depicted by solid lines and a light collecting position depicted by dashed lines is smaller in the case in which the object distance in the side view direction is short as compared to that in the case in which the object distance in the side view direction is long.

The light collecting position depicted by dashed lines is a position when the light beam is not refracted at the inner peripheral surface 2a. This position indicates a light collecting position at the meridional cross-section. From FIG. 7A and FIG. 7B, it is evident that the predetermined difference in positions in the case in which the object distance in the side view direction is short is small as compared to that in the case in which the object distance in the side view direction is long.

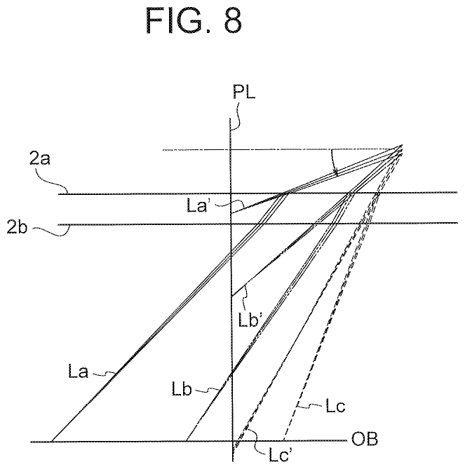

FIG. 8 is a diagram showing an appearance of a light beam at the meridional cross section. A position of a plane PL indicates a position of a best plane at the meridional cross section. At the position of the best plane, the spot diagram becomes the smallest. The plane PL is the best plane when the object distance is 2 mm and the angle of view is 20°.

As described heretofore, as the object distance in the side view direction becomes larger, the amount of occurrence of astigmatism becomes large. Therefore, in the side view direction, it is desirable that the object plane be positioned near the outer peripheral surface. Since the object distance in the side view direction is associated with a size of curvature of the image plane, it is desirable to make the value of Petzval sum appropriate.

In the endoscope of the present embodiment, formation of the optical image of the object is carried out via liquid. Therefore, it is desirable to determine the value of Petzval sum in the image forming optical system 4 up on taking into consideration a fact that the optical image of the object is formed via liquid.

For such reasons, in the endoscope of the present embodiment, the following conditional expression (1) is satisfied:

−10<P′<−0.8 (1),

where,

P′ denotes Petzval sum, and is expressed by the following expression,

$${P}^{′} = {n}^{′} {\sum }_{i = 1}^{k} \frac{1}{{r}_{i}} ( \frac{1}{{n}_{i}^{′}} - \frac{1}{n} ) ,$$

rᵢ denotes a radius of curvature of an i^{th} transmitting surface,

n′ᵢ denotes a refractive index at an emergence side of the i^{th} transmitting surface,

nᵢ denotes a refractive index at an incidence surface of the i^{th} transmitting surface,

n′ denotes a refractive index of an image space,

i denotes a number of a transmitting surface, and

k denotes the total number of transmitting surfaces.

By satisfying conditional expression (1), it is possible to make the value of Petzval sum appropriate. When conditional expression (1) is satisfied, it is possible to make the image forming optical system to have the curvature of field suitable for a shape of an object, while maintaining the astigmatism that occurs at the inner peripheral surface of the circular cylindrical member at the minimum. Consequently, at the time of liquid observation, it is possible to form a sharp optical image in the side view direction. As a result, according to the endoscope of the present embodiment, it is possible to observe clearly an inner surface in lumen for example.

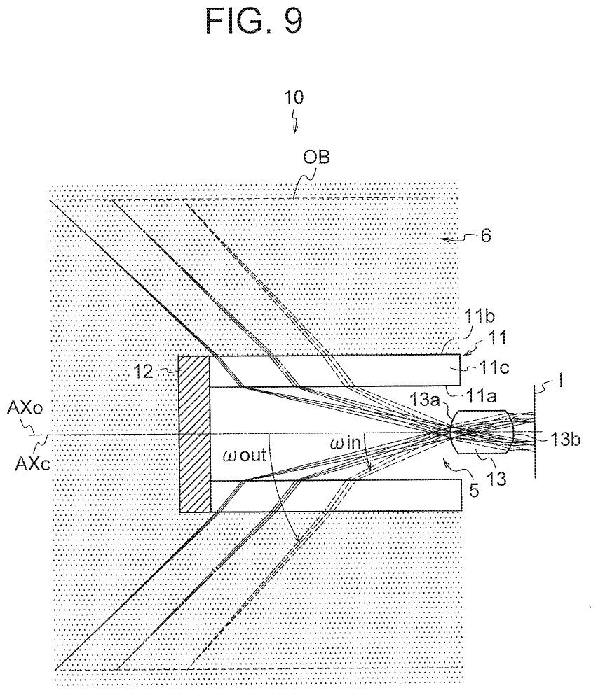

Another optical unit of the present embodiment is shown in FIG. 9. An optical unit 10 includes a circular cylindrical member 11, a distal end member 12, and an image forming optical system 13. The circular cylindrical member 11 has an inner peripheral surface 11a and an outer peripheral surface 11b. A space between the inner peripheral surface 11a and the outer peripheral surface 11b is filled with a transparent material 2c having a refractive index higher than 1. The distal end member 12 is positioned at one end of the circular cylindrical member 11.

The image forming optical system 13 is disposed at an interior of the circular cylindrical member 11 such that an optical axis AXo and a central axis AXc are aligned. An object plane OB and an image plane I are conjugate due to the image forming optical system 13. The object plane OB is depicted by a dashed line. The object plane OB is positioned at an outer side of the outer peripheral surface 11b.

The image forming optical system 13 is formed by one single lens. The image forming optical system 13 has a transmitting surface 13a and a transmitting surface 13b. In such manner, the image forming optical system 13 is formed by only transmitting surfaces. In the image forming optical system 13, an aperture stop is located on the transmitting surface 13a.

The transmitting surface 13a and the transmitting surface 13b are disposed such that a normal of a plane at a point intersecting with the optical axis AXo is aligned with the optical axis AXo. In such manner, all the transmitting surfaces of the image forming optical system 13 are disposed such that the normal of the plane at the point intersecting with the optical axis AXo is aligned with the optical axis AXo.

The circular cylindrical member 11 is positioned in a side view direction. The distal end member 12 is positioned in the direct view direction. The circular cylindrical member 11 is formed of a transparent material, and the distal end member 12 is formed of an opaque material. Therefore, in the optical unit 10, an image in the side view direction is formed on the image plane I via the circular cylindrical member 11, but an image in the direct view direction is not formed. When the distal end member 12 is formed of a transparent material, the image in the direct view direction is formed.

A first space 5 is a space formed by a space positioned at an inner side of the circular cylindrical member 11 and a space positioned at an inner side of the distal end member 12. The first space 5 is filled with air. A second space 6 is a space formed by a space positioned at an outer side of the circular cylindrical member 11 and a space positioned at an outer side of the distal end member 12. The second space 6 is filled with water. The object plane OB being located in the second space 6, formation of an optical image of the object is carried out via water.

The object plane OB in the side view direction is a circularly cylindrical surface similar to the outer peripheral surface 11b. Therefore, in the image forming optical system 13, an image of a circular cylindrical surface is formed on a flat surface.

The outer peripheral surface 11b is a circular cylindrical surface of which a diameter is 1 mm. The object plane OB is a circular cylindrical surface of which a diameter is 3 mm. A half angle of view coin in the first space 5 is ±24.9°, and a half angle of view ωout in the second space 6 is ±53.3°

In such manner, in the optical unit 10, it is possible to achieve an angle of view in the second space 6, wider than an angle of view in the first space 5 of the image forming optical system 13. Such effect of widening the angle of view is due to the fact that the first space 5 is air and the second space 6 is water.

In a conventional endoscope having thin diameter, a structure of an optical unit disposed at a distal end of an insertion portion was complicated. Consequently, it was difficult to insert the distal end into a thin tube of a diameter of 10 mm or less. According to the endoscope of the present embodiment, it is possible to insert the distal end into a thin tube. Moreover, due to the abovementioned effect of widening the angle of view, it is possible to form an image of an inner wall of a thin tube in all directions by being transmitted through the circular cylindrical member.

In the endoscope of the present embodiment, it is preferable that the following conditional expression (2) be satisfied:

0.1 mm<f<0.8 mm (2),

where,

f denotes a focal length of the image forming optical system.

In the endoscope of the present embodiment, for making the optical unit small-sized, the focal length of the image forming optical system has been made extremely short. For instance, in an example 2 to be described later, the focal length of the image forming optical system is 0.296 mm. In the image forming optical system of the example 2, for instance, it is possible to observe an inner surface of a thin tube of which a diameter is 3 mm.

Conditional expression (2) is a conditional expression necessary for causing the curvature of field such that a sharp image is formed, even in a case in which the object distance in the side view direction is short. As mentioned above, Petzval sum is an index for expressing the curvature of field. The value of Petzval sum depends on the focal length of the image forming optical system. Therefore, it is desirable to satisfy conditional expression (2).

In a case of falling below a lower limit value of conditional expression (2), the amount of occurrence of the curvature of field becomes excessively small. In this case, it is not possible to form a sharp optical image in the periphery of an observation range. In a case of exceeding an upper limit value of conditional expression (2), the amount of occurrence of the curvature of field becomes excessively large. In this case, it is not possible to form a sharp optical image at an object point located far from the optical axial direction.

In the endoscope of the present embodiment, it is preferable that the following conditional expression (3) be satisfied:

θout<θin (3),

where,

θin is an angle made by a principal light ray and a normal of the inner peripheral surface, in a first space (however, θ≠0),

θout is an angle made by the principal light ray and a normal of the outer peripheral surface, in a second space,

the first space is a space between the image forming optical system and the inner peripheral surface,

the second space is a space at an outer side of the circular cylindrical member, and

the principal light ray is a principal light ray from an object point of a center when the circular cylindrical member is measured in an optical axial direction.

In the endoscope of the present embodiment, an image of the object plane positioned in the side view direction is formed via a circular cylindrical body. The principal light ray in conditional expression (3) is a principal light ray that reaches the aperture stop of the image forming optical system from a center of the range of the object plane on which an image can be formed.

In the endoscope of the present embodiment, it is preferable that the following conditional expression (4) be satisfied:

1<R2/R1<5 (4),

where,

R1 denotes a radius of curvature of the inner peripheral surface, and

R2 denotes a radius of curvature of the outer peripheral surface.

In a case of falling below a lower limit value of conditional expression (4), a thickness of the circular cylindrical member becomes excessively thin. This leads to a lack of strength of the circular cylindrical member. In a case of exceeding an upper limit value of conditional expression (4), the amount of occurrence of astigmatism at the circular cylindrical member becomes excessively large. Consequently, in the image forming optical system, it is not possible to correct the astigmatism.

In the endoscope of the present embodiment, it is preferable that the following conditional expression (5) be satisfied:

1≤OB/R2<10 (5),

where,

OB denotes a distance from the optical axis up to the object plane, in a plane orthogonal to the optical axis, and

R2 denotes a curvature of the outer peripheral surface.

In a case of falling below a lower limit value of conditional expression (5), it is not possible to form a sharp optical image in the side view direction. In a case of exceeding an upper limit value of conditional expression (5), the astigmatism becomes excessively large. In this case, a resolution performance in a sagittal direction is degraded. Consequently, it is not possible to form a sharp optical image in the side view direction.

In the endoscope of the present embodiment, it is preferable that the image forming optical system include in order from the distal end member side, a first positive lens and a second positive lens, a first predetermined surface be a lens surface on the image plane side of the first positive lens, a second predetermined surface be a lens surface on the distal end member side of the second positive lens, the first predetermined surface be a convex surface directed toward the second positive lens, and the second predetermined surface be a convex surface toward the first positive lens.

By bringing convex surfaces having a positive refractive power face-to-face, it is possible to generate easily the curvature of field with an appropriate quantity.

As a lens in which degree of difficulty of fabrication is low, and which can be fabricated with high accuracy, a ball lens is known. However, in the ball lens, the astigmatism occurs largely on a minus side. Moreover, as mentioned above, in the in-liquid observation via the circular cylindrical member, the astigmatism occurs in the minus direction. Consequently, when the ball lens is used in the image forming optical system, the astigmatism occurs further largely.

Therefore, by bringing the convex surfaces having a positive refractive power face-to-face, it is possible to generate the astigmatism on a plus side. In other words, it is possible to make the amount of astigmatism that occurs on the minus side smaller as compared to that in the ball lens. As a result, it is possible to make the amount of occurrence of the astigmatism small wholly.

It is possible to realize the convex surface having a positive refractive power by a planoconvex lens. It is possible to achieve the planoconvex lens by grinding one side of the ball lens to a flat surface. As mentioned above, in the ball lens, the degree of difficulty of fabrication is low, and it can be fabricated with high accuracy. Therefore, the planoconvex lens can also be fabricated easily and with high accuracy.

In the endoscope of the present embodiment, it is preferable that the following conditional expressions (6) and (7) be satisfied:

0.5<ϕ1/ϕ<5.0 (6), and

0.1<ϕ2/ϕ<2.0 (7),

where,

ϕ denotes a refractive power of the image forming optical system,

ϕ1 denotes a refractive power of the first predetermined surface, and

ϕ2 denotes a refractive power of the second predetermined surface.

In the side view direction, when the object distance becomes long, the astigmatism which occurs in the minus direction becomes large. Therefore, it is preferable that the image forming optical system have a lens surface which generates the astigmatism in the plus direction. As mentioned above, by bringing the convex surfaces having a positive refractive power face-to-face, it is possible to generate the astigmatism in the plus direction.

In a case of falling below both a lower limit value of conditional expression (6) and a lower limit value of conditional expression (7), it is not possible to generate the astigmatism on the plus side. In a case of exceeding both an upper limit value of conditional expression (6) and an upper limit value of conditional expression (7), the astigmatism occurs largely on the plus side. Consequently, it is not possible to have a wide angle of view that enables to form a sharp optical image.

In the endoscope of the present embodiment, it is preferable that the image forming optical system include a planoconvex lens.

In the endoscope of the present embodiment, a diameter of the image forming optical system is extremely small. Particularly, when the diameter becomes 1 mm or less, since the fabrication of a lens becomes difficult, the cost becomes high. Moreover, assembling also becomes difficult. However, it is possible to achieve the planoconvex lens by grinding one side of the ball lens to a flat surface for example. In such manner, the fabrication of the planoconvex lens being easy, it is possible to realize an image forming optical system with a small diameter at an inexpensive price.

In the endoscope of the present embodiment, it is preferable that the image forming optical system include a ball lens.

The ball lens can be used as a lens as it is. Therefore, it is possible to realize an image forming optical system with a small diameter at an inexpensive price.

In the endoscope of the present embodiment, it is preferable that the image forming optical system include a gradient index lens.

In the gradient index lens, it is possible to make both end surfaces flat surfaces. Consequently, assembling of the optical system becomes easy.

In the endoscope of the present embodiment, it is preferable that the image forming optical system be disposed at a distal end of an insertion portion of the endoscope, the distal end of the insertion portion have a connecting portion, the circular cylindrical member have a connecting portion on the other end, and the circular cylindrical member be attached to and detached from the insertion portion via the two connecting portions.

According to the endoscope of the present embodiment, in spite of a diameter of the insertion portion being thin, at the time of the in-liquid observation, it is possible to acquire a sharp image in the side view direction.

The connecting portion being provided to each of the circular cylindrical member and the insertion portion of the endoscope, it is possible to attach and detach the circular cylindrical member to and from the insertion portion. The distal end member is located at one end of the circular cylindrical member. Therefore, it is possible to attach and detach the distal end member as well, to and from the insertion portion. In such manner, according to the endoscope of the present embodiment, it is possible to replace both the circular cylindrical member and the distal end member.

It is possible to form a cover unit by the circular cylindrical member and the distal end member. With respect to the circular cylindrical member and the distal end member, it is possible to change a shape, a size, a thickness, and a material, for example. Therefore, it is possible to prepare a plurality of cover units with different specifications. By doing so, it is possible to carry out observation with a cover unit appropriate for observation.

In the endoscope of the present embodiment, it is preferable that the image forming optical system be disposed on a distal end of the insertion portion of the endoscope, and the circular cylindrical member be fixed to the distal end of the insertion portion all the time.

According to the endoscope of the present embodiment, in spite of the diameter of the insertion portion being thin, at the time of the in-liquid observation, it is possible to acquire a sharp image in the side view direction.

Moreover, the circular cylindrical member being fixed to the distal end of the insertion portion all the time, it is possible to maintain a high airtightness. Consequently, according to the endoscope of the present embodiment, it is possible to protect the image forming optical system from dirt and the like.

Examples of an image forming optical system used for an endoscope will be described below in detail by referring to the accompanying diagrams. However, the present disclosure is not restricted to the examples described below.

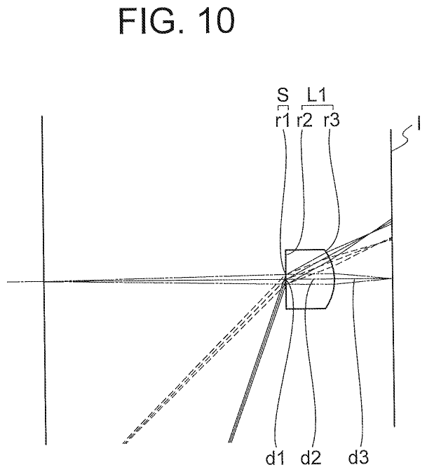

A lens cross-sectional view of an image forming optical system of an example 1 is shown in FIG. 10. The image forming optical system of the example 1 includes a planoconvex lens L1. An aperture stop S is disposed on an object-side surface of the planoconvex lens L1.

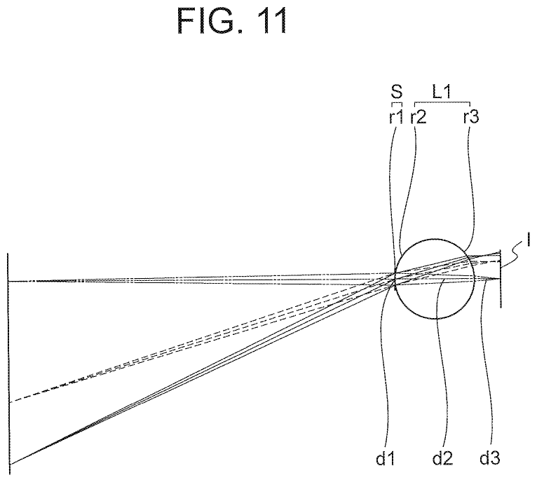

A lens cross-sectional view of the image forming optical system of the example 2 is shown in FIG. 11. The image forming optical system of the example 2 includes a biconvex lens L1. An aperture stop S is disposed on an object-side surface of the biconvex lens L1. It is preferable to make the biconvex lens L1 a ball lens.

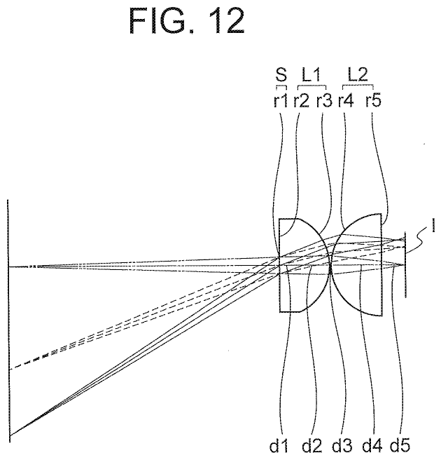

A lens cross-sectional view of an image forming optical system of an example 3 is shown in FIG. 12. The image forming optical system of the example 3 includes a planoconvex lens L1 and a planoconvex lens L2. An aperture stop S is disposed on an object-side surface of the planoconvex lens L1.

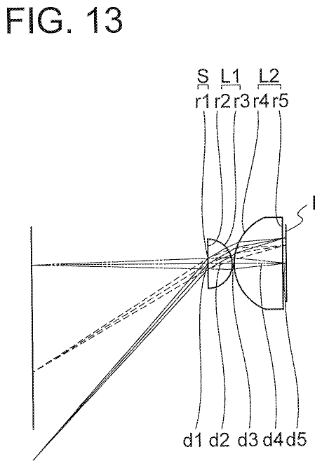

A lens cross-sectional view of an image forming optical system of an example 4 is shown in FIG. 13. The image forming optical system of the example 4 includes a planoconvex lens L1 and a planoconvex lens L2. An aperture stop S is disposed on an object-side surface of the planoconvex lens L1.

Numerical data of each example described above is shown below. In Surface data, r denotes radius of curvature of each lens surface, d denotes a distance between respective lens surfaces, nd denotes a refractive index of each lens for a d-line, and νd denotes an Abbe number for each lens.

Moreover, in various data, f is a focal length of the overall system, ω is a half angle of view, IH is an image height, and φap is a diameter of a stop. In the image forming optical system of each example, an image in the direct view direction is formed in a circular shape. An image in the side view direction is formed at an outer side of the image in the direct view direction. Therefore, the image in the side view direction is formed in an annular shape. The image height IH indicates an outer diameter of the annular-shaped image.

### Example 1

Unit mm
Surface data

| Surface no. | r | d | nd | νd |
|---|---|---|---|---|
| Object plane | ∞ | 2.300 | | |
| 1(Stop) | ∞ | 0.000 | | |
| 2 | ∞ | 0.500 | 2.0033 | 28.3 |
| 3 | −0.500 | 0.611 | | |
| Image plane | ∞ | | | |

Various data

| | |
|---|---|
| f | 0.494 |
| ω | 70° |
| IH | 1.17 |
| φap | 0.1 |

### Example 2

Unit mm
Surface data

| Surface no. | r | d | nd | νd |
|---|---|---|---|---|
| Object plane | ∞ | 2.000 | | |
| 1(Stop) | ∞ | 0.000 | | |
| 2 | 0.202 | 0.405 | 1.5163 | 64.1 |
| 3 | −0.202 | 0.137 | | |
| Image plane | ∞ | | | |

Various data

| | |
|---|---|
| f | 0.296 |
| ω | 25° |
| IH | 0.26 |
| φap | 0.06 |

### Example 3

Unit mm
Surface data

| Surface no. | r | d | nd | νd |
|---|---|---|---|---|
| Object plane | ∞ | 1.400 | | |
| 1(Stop) | ∞ | 0.000 | | |
| 2 | ∞ | 0.258 | 1.5163 | 64.1 |
| 3 | −0.258 | 0.010 | | |
| 4 | 0.258 | 0.258 | 1.5163 | 64.1 |
| 5 | ∞ | 0.124 | | |
| Image plane | ∞ | | | |

Various data

| | |
|---|---|
| f | 0.252 |
| ω | 32° |
| IH | 0.26 |
| φap | 0.06 |

### Example 4

Unit mm
Surface data

| Surface no. | r | d | nd | νd |
|---|---|---|---|---|
| Object plane | ∞ | 1.000 | | |
| 1(Stop) | ∞ | 0.000 | | |
| 2 | ∞ | 0.125 | 1.5163 | 64.1 |
| 3 | −0.125 | 0.010 | | |
| 4 | 0.250 | 0.250 | 1.5163 | 64.1 |
| 5 | ∞ | 0.018 | | |
| Image plane | ∞ | | | |

Various data

| | |
|---|---|
| f | 0.163 |
| ω | 47.4° |
| IH | 0.26 |
| φap | 0.06 |

Next, values of conditional expressions in each example are given below. ‘-’ (hyphen) indicates that there is no corresponding arrangement.

| | Example1 | Example2 | Example3 | Example4 |
|---|---|---|---|---|
| (1)P′ | −1.002 | −3.366 | −2.640 | −1.362 |
| (2)f | 0.494 | 0.296 | 0.252 | 0.163 |
| (4)R2/R1 | 1.20 | 1.20 | — | — |
| (5)OB/R2 | 1.17 | 1.17 | — | — |
| (6)ϕ1/ϕ | — | — | 0.504 | 0.674 |
| (7)ϕ2/ϕ | — | — | 0.504 | 0.337 |

Values of parameters are given below.

| | Example1 | Example2 | Example3 | Example4 |
|---|---|---|---|---|
| R1 | — | — | 0.50 | 0.50 |
| R2 | — | — | 0.60 | 0.60 |
| OB | — | — | 0.70 | 0.70 |
| ϕ1 | — | — | 2.001 | 4.130 |
| ϕ2 | — | — | 2.001 | 2.065 |
| ϕ = 1/F | 2.023 | 3.375 | 3.971 | 6.131 |

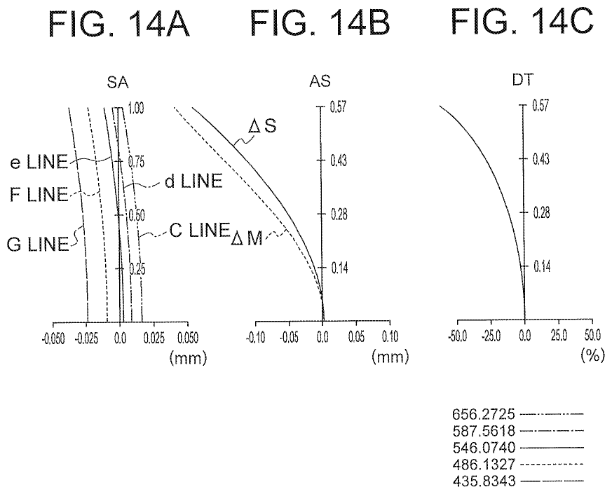

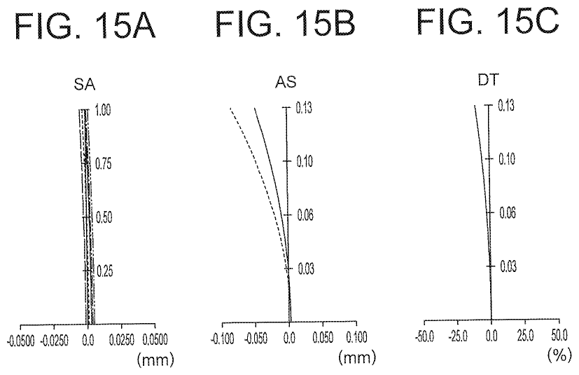

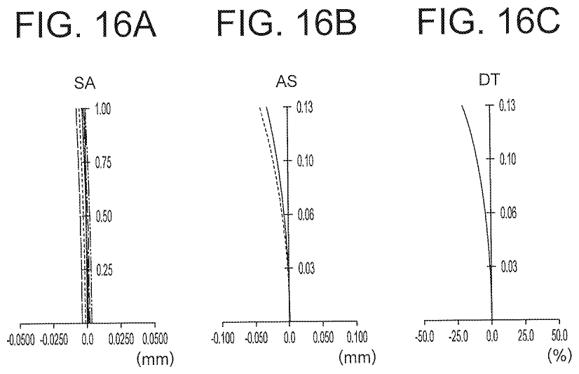

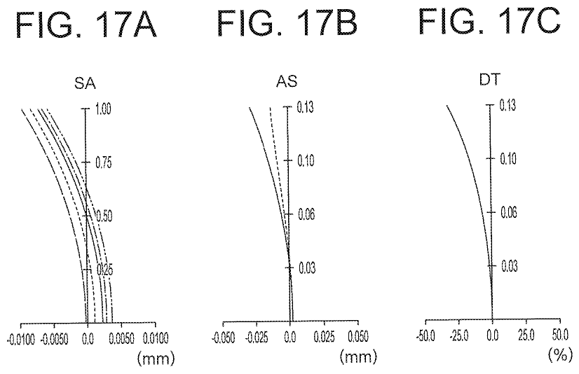

Aberration diagrams of each example will be described below. FIG. 14A, FIG. 15A, FIG. 16A, and FIG. 17A show a spherical aberration (SA). FIG. 14B, FIG. 15B, FIG. 16B, and FIG. 17B show an astigmatism (AS). FIG. 14C, FIG. 15C, FIG. 16C, and FIG. 17C show a distortion (DT).

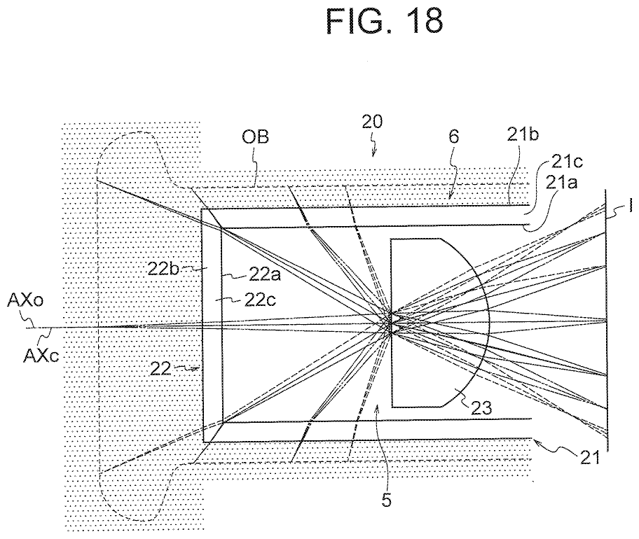

Examples of the optical unit will be shown. FIG. 18 is a diagram showing a first example of the optical unit. An optical unit 20 includes a circular cylindrical member 21, a distal end member 22, and an image forming optical system 23. The circular cylindrical member 21 has an inner peripheral surface 21a and an outer peripheral surface 21b. A space between the inner peripheral surface 21a and the outer peripheral surface 21b is filled with a transparent material 21c having a refractive index higher than 1.

The distal end member 22 is a plane parallel plate, and is positioned at one end of the circular cylindrical member 21. The distal end member 22 has an inner surface 22a and an outer surface 22b. A space between the inner surface 22a and the outer surface 22b is filled with a transparent material 22c having a refractive index higher than 1.

The image forming optical system 23 is disposed at an interior of the circular cylindrical member 21 such that an optical axis AXo and a central axis AXc are aligned. An object plane OB and an image plane I are conjugate due to the image forming optical system 23. The object plane OB is depicted by a dashed line. The object plane OB is positioned at an outer side of the outer peripheral surface 21b and an outer side of the outer surface 22b.

The image forming optical system of the example 1 is used for the image forming optical system 23. In the optical unit 20, an image in the side view direction and an image in the direct view direction are formed on the image plane I.

Specifications of the optical unit 20 are shown below. A thickness of the circular cylindrical member is a thickness of the material 21c, and a refractive index of the circular cylindrical member is a refractive index of the material 21c. A thickness of the distal end member is a thickness of the material 22c, and a refractive index of the distal end member is a refractive index of the material 22c.

An object distance 1 and an object distance 2 are distances in the direct view direction. The object distance 1 is a distance from an aperture stop of the image forming optical system 23 to the object plane OB. The object distance 2 is a distance from outer surface 22b to the object plane OB. An object distance 3 is a distance in the side view direction. The object distance 3 is a distance from an optical axis to the object plane OB, on a plane orthogonal to the optical axis of the image forming optical system 23.

diameter of the inner peripheral surface: 1 mm

diameter of the outer peripheral surface: 1.2 mm

thickness of the circular cylindrical member: 0.1 mm

refractive index of the circular cylindrical member: 1.51633

thickness of the distal end member: 0.1 mm

refractive index of the distal end member: 1.51633

refractive index of the first space: 1

refractive index of the second space: 1.33

object distance 1: 1.5 mm

object distance 2: 0.535 mm

object distance 3: 0.7 mm

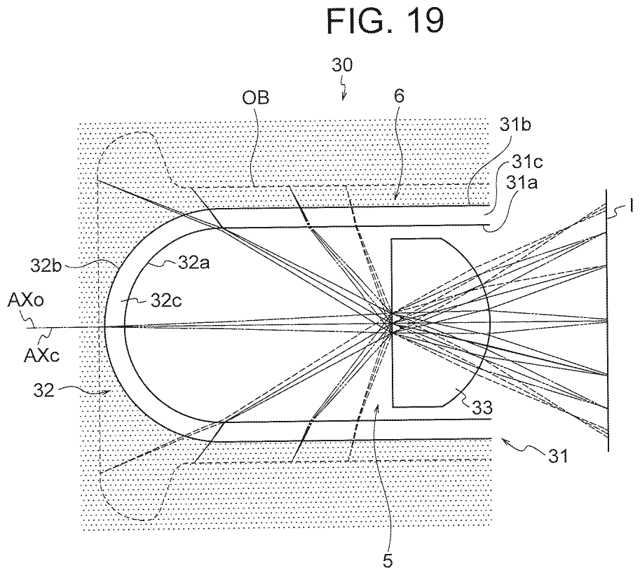

FIG. 19 is a diagram showing a second example of the optical unit. An optical unit 30 includes a circular cylindrical member 31, a distal end member 32, and an image forming optical system 33. The circular cylindrical member 31 has an inner peripheral surface 31a and an outer peripheral surface 31b. A space between the inner peripheral surface 31a and the outer peripheral surface 31b is filled with a transparent material 31c having a refractive index higher than 1.

The distal end member 32 is a semispherical plate, and is positioned at one end of the circular cylindrical member 31. The distal end member 32 has an inner surface 32a and an outer surface 32b. A space between the inner surface 32a and the outer surface 32b is filled with a transparent material 32c having a refractive index higher than 1.

The image forming optical system 33 is disposed at an interior of the circular cylindrical member 31 such that an optical axis AXo and a central axis AXc are aligned. An object plane OB and an image plane I are conjugate due to the image forming optical system 33. The object plane OB is depicted by a dashed line. The object plane OB is positioned at an outer side of the outer peripheral surface 31b and an outer side of the outer surface 32b.

The image forming optical system of the example 1 is used for the image forming optical system 33. In the optical unit 30, an image in the side view direction and an image in the direct view direction are formed on the image plane I. Specifications of the optical unit 30 are shown below.

diameter of the inner peripheral surface: 1 mm

diameter of the outer peripheral surface: 1.2 mm

thickness of the circular cylindrical member: 0.1 mm

refractive index of the circular cylindrical member: 1.51633

radius of curvature of the inner surface: 0.5 mm

radius of curvature of the outer surface: 0.6 mm

thickness of the distal end member: 0.1 mm

refractive index of the distal end member: 1.51633

refractive index of the first space: 1

refractive index of the second space: 1.33

object distance 1: 1.5 mm

object distance 2: 0.535 mm

object distance 3: 0.7 mm

Examples of the insertion portion of the endoscope of the present embodiment (hereinafter, referred to as ‘insertion portion of the present embodiment’) will be described below. In the following examples, an image sensor is disposed on an image plane of the image forming optical system, and an optical image formed by the image forming optical system is captured by the image sensor. However, the image sensor may not be disposed on the image plane of the image forming optical system. For instance, an optical image formed by the image forming optical system may be transmitted by an image fiber (fiber bundle). Moreover, the optical image formed by the image forming optical system may be observed visually.

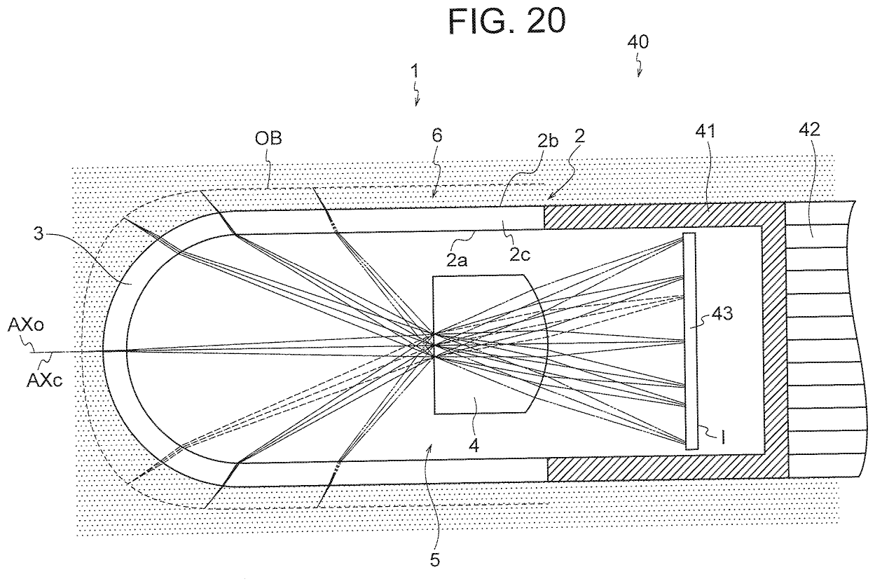

FIG. 20 is a diagram showing a first example of the insertion portion of the present embodiment. Same reference numerals are assigned to components that are same as in FIG. 1, and description thereof is omitted.

An insertion portion 40 includes the optical unit 1, a holding member 41, and a guide wire 42. The distal end member 3 is disposed at one end of the circular cylindrical member 2, and the holding member 41 is disposed at the other end. A hermetically sealed space is formed by the circular cylindrical member 2, the distal end member 3, and the holding member 41.

The image forming optical system 4 is disposed in the hermetically sealed space. An image sensor 43 is disposed on an image plane I. It is possible to acquire an image of an optical image by the image sensor 43. The image forming optical system 4 and the image sensor 43 are fixed near a distal end of the insertion portion 40. Accordingly, it is not possible to remove the image forming optical system 4 and the image sensor 43 from the insertion portion 40.

The holding member 41 and the guide wire 42 form the insertion portion 40. The holding member 41 is positioned at the front end of the guide wire 42. The holding member 41 is made of a metal for example. The guide wire 42 is connected to one end of the holding member 41. The guide wire 42 has a flexible structure. Accordingly, it is possible to put the endoscope into and take out from a thin tube easily.

The circular cylindrical member 2 and the holding member 41 are fixed by an adhesive for example. Accordingly, the circular cylindrical member 2 is fixed to the distal end of the insertion portion 40 all the time. In such manner, in the first example, it is not possible to attach and detach the circular cylindrical member 2 to and from the insertion portion 40.

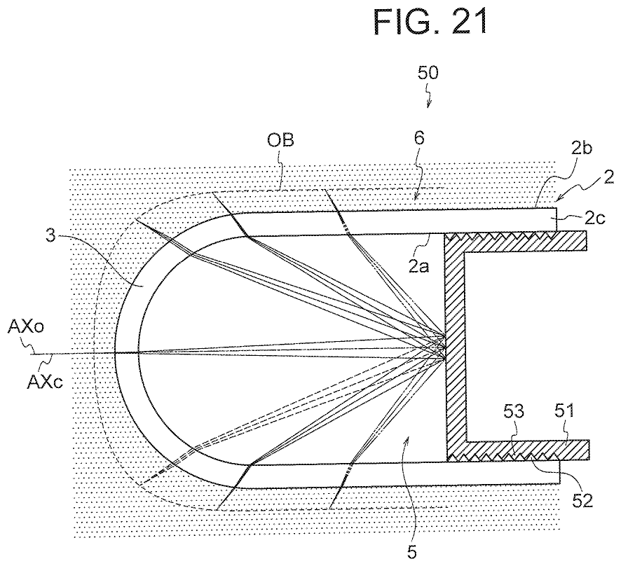

FIG. 21 is a diagram showing a second example of the insertion portion of the present embodiment. Same reference numerals are assigned to components that are same as in FIG. 1, and description thereof is omitted. The image forming optical system and the image sensor are omitted in the diagram, and light rays are illustrated in the diagram.

An insertion portion 50 includes the circular cylindrical member 2, the distal end member 3, and a holding member 51. The image forming optical system and the image sensor are fixed near a distal end of the insertion portion 50. Accordingly, it is not possible to remove the image forming optical system and the image sensor from the insertion portion 50.

The holding member 51 forms the insertion portion 50. The holding member 51 is positioned at the distal end of the insertion portion 50. The circular cylindrical member 2 has a connecting portion 52 at the other end. The holding member 51 also has a connecting portion 53. Accordingly, in the second example, it is possible to attach and detach the circular cylindrical member 2 to and from the insertion portion 50 via the connecting portion 52 and the connecting portion 53. A screw for instance, may be used for the connecting portion 52 and the connecting portion 53.

The distal end member 3 is located at one end of the circular cylindrical member 2. Accordingly, it is possible to attach and detach the distal end member 3 as well, to and from the insertion portion 50. In such manner, in the second example, it is possible to replace both the circular cylindrical member 2 and the distal end member 3. It is possible to form a cover unit by the circular cylindrical member and the distal end member, and to attach and detach the cover unit to and from the insertion portion.

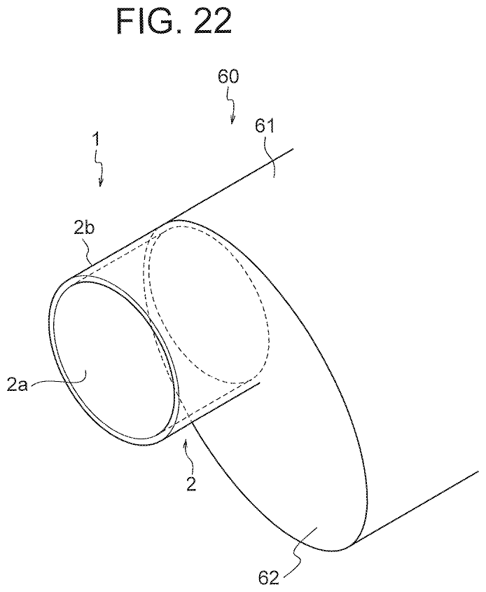

FIG. 22 is a diagram showing a third example of the insertion portion of the present embodiment. Same reference numerals are assigned to components that are same as in FIG. 1, and description thereof is omitted. The image forming optical system and the image sensor are omitted in the diagram.

An insertion portion 60 includes the optical unit 1 and a holding member 61. The image forming optical system is disposed in the optical unit 1. The image forming optical system and the image sensor are fixed to the insertion portion 60. Accordingly, it is not possible to remove the image forming optical system and the image sensor from the insertion portion 60.

In the third example, a diameter of the optical unit 1 is smaller than a diameter of the holding member 61. Moreover, an optical axis of the image forming optical system is not aligned with a central axis of the holding member 61. In other words, the optical unit 1 is disposed in a peripheral portion of the holding member 61.

Therefore, the holding member 61 has a flat portion 62. So, it is possible to dispose an illuminating optical system on the flat portion 62. Or, it is possible to provide an opening to the flat portion 62, for putting in and taking out a treatment tool.

It is possible to fix the circular cylindrical member 2 by an adhesive to the insertion portion 60 all the time. Or, it is possible to attach and detach the circular cylindrical member 2 to and from the insertion portion 60 by a screw for example.

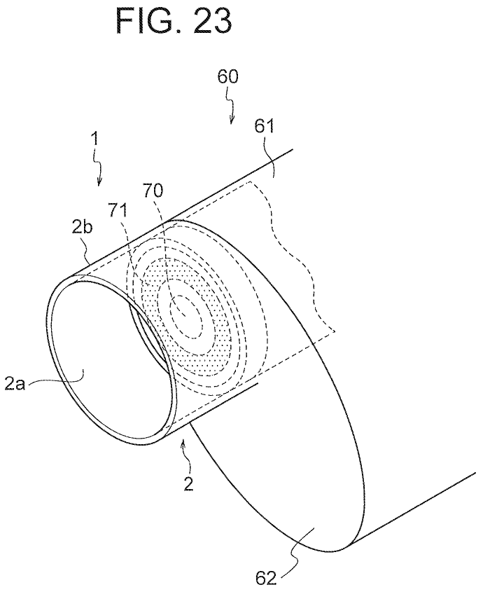

FIG. 23 is a diagram showing an arrangement example of the illuminating optical system. Same reference numerals are assigned to components that are same as in FIG. 22, and description thereof is omitted. A specific arrangement of the image forming optical system, and the image sensor are omitted in the diagram.

The insertion portion 60 includes the optical unit 1 and the holding member 61. An image forming optical system 70 and an illuminating optical system 71 are disposed in the optical unit 1. A shape of the illuminating optical system 71 is an annular shape. The illuminating optical system 71 is positioned at an outer side of the image forming optical system 70.

An object plane is illuminated by illumination light from the illuminating optical system 71. Light from the object plane is focused at an image plane by the image forming optical system 70. In such manner, an optical image of an object is formed on the image plane.

In the description made heretofore, the image forming optical system is treated as an optical system for forming an optical image of an object. However, it is possible to use the image forming optical system as a scanning optical system which makes the illuminating light scan.

For the image forming optical system 70, it is possible use the image forming optical system 4 shown in FIG. 1 for instance. As shown in FIG. 1, light from one point on the object plane is focused at one point on the image plane I. This signifies that, when a light source is disposed at one point on the image plane I, light emerged from the light sources is focused at one point on the object plane OB.

Therefore, a point light source for instance is disposed at a position on the image plane I. By making such arrangement, it is possible to illuminate one point on the object plane OB. Moreover, by receiving light from the one point which is illuminated, it is possible to acquire information of one point of the object plane OB. For receiving light from the object plane OB, a light receiving element is to be disposed at a location of the illuminating optical system 71.

Furthermore, by moving the point light source, it is possible to acquire information of the overall object plane OB. For the movement of the point light source, for example, an end portion of one optical fiber is to be moved on the image plane. It is possible to realize the movement of the end portion of the optical fiber by disposing an actuator near the end portion of the optical fiber for example. It is possible to make a movement locus of the optical fiber spiral for example.

The light source to be disposed at the position on the image plane may be any light source provided that the light source can be deemed as a point light source. When a size of a light emerging surface of the optical fiber is about a size that can be deemed as a point light source, the light emerging surface can also be called a point light source. As the optical fiber, it is possible to use a single mode fiber for example.

A light emerging surface of the fiber bundle may be disposed at a position of the image plane. In the fiber bundle, a plurality of optical fibers is bundled into one. By changing the optical fiber that makes the illuminating light incident, it is possible to realize the movement of the point light source without moving the optical fiber.

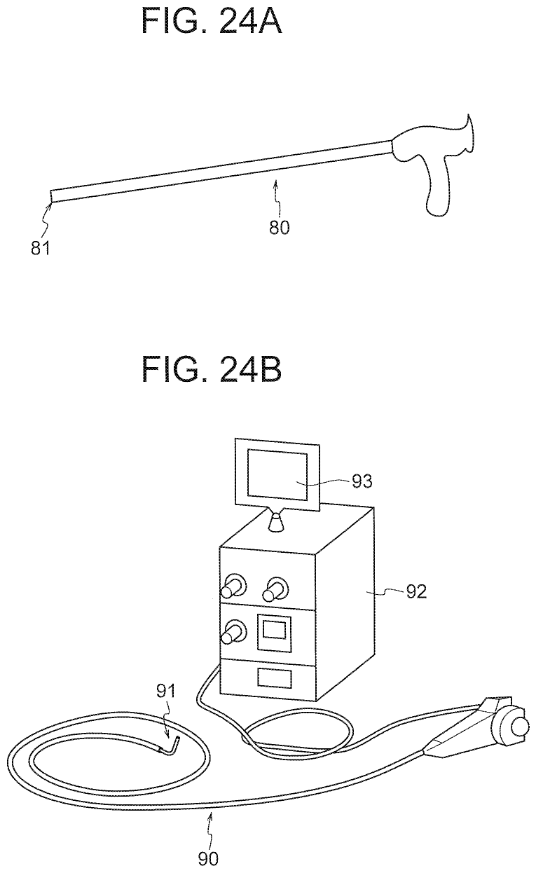

FIG. 24A and FIG. 24B are diagrams showing examples of the endoscope. FIG. 24A is a diagram illustrating a rigid endoscope and FIG. 24B is a diagram illustrating a soft endoscope.

AS shown in FIG. 24A, an optical unit 81 is disposed at a distal end of an insertion portion of an endoscope 80. It is possible to use the optical unit of the present embodiment for the optical unit 81. Accordingly, it is possible to acquire an image in the side view direction in all directions. Consequently, it is possible to observe various parts from angles different from those in the conventional endoscopes.

Moreover, as shown in FIG. 24B, an optical unit 91 is disposed at a distal end of an insertion portion of an endoscope 90. It is possible to use the optical unit of the present embodiment for the optical unit 91. Accordingly, it is possible to acquire an image in the side view direction in all directions. Consequently, it is possible to observe various parts from angles different from those in the conventional endoscopes.

It is possible to display an acquired image on a display unit 93 via an image processing unit 92. In the image processing unit 92, it is possible to carry out various image processing.

In the optical unit 81 and the optical unit 91, both the circular cylindrical member and the distal end member may be replaceable with respect to the insertion portion, or may be fixed to the insertion portion all the time.

The image forming optical system may be fixed to the insertion portion. However, the image forming optical system may be made to be attachable and detachable to and from the insertion portion with the circular cylindrical member and the distal end member. When such arrangement is made, it is possible to attach and detach the optical unit to and from the insertion portion. The optical unit may satisfy at least one of conditional expressions (1) to (7).

The optical unit being attachable and detachable to and from the insertion portion, the replacement of the optical unit becomes possible. For example, when a plurality of optical units having different optical specifications is prepared, it is possible to carry out observation by an optical unit which is suitable for observation.

Moreover, an arrangement may be made such that the optical unit and the image sensor are integrated, and can be attached or detached to and from the insertion portion.

According to the present disclosure, it is possible to provide an endoscope which enables to form a sharp optical image in the side view direction at the time of in-liquid observation, while having a thin diameter.

As described heretofore, the present disclosure is suitable for an endoscope which enables to form a sharp optical image in the side view direction at the time of in-liquid observation, while having a thin diameter.

What is claimed is:

1. An endoscope comprising:
a circular cylindrical member;
a distal end member; and
an image forming optical system,
wherein:
the circular cylindrical member has an inner peripheral surface and an outer peripheral surface,
a space between the inner peripheral surface and the outer peripheral surface is filled with a transparent material having a refractive index higher than 1,
the distal end member is positioned at one end of the circular cylindrical member,
the image forming optical system is disposed at an interior of the circular cylindrical member such that an optical axis of the image forming optical system and a central axis of the circular cylindrical member are aligned or become parallel,
due to the image forming optical system, an object plane located at an outer side of the outer peripheral surface and an image plane of the image forming optical system become conjugate,
the image forming optical system includes only transmitting surfaces,
all the transmitting surfaces are disposed such that a normal of a plane at a point intersecting with the optical axis is aligned with the optical axis,
the image forming optical system has a curvature of field, and
the following conditional expression (1) is satisfied:
−10<P′<−0.8 (1),
where,
P′ denotes Petzval sum, and is expressed by the following expression,
$${P}^{′} = {n}^{′} {\sum }_{i = 1}^{k} \frac{1}{{r}_{i}} ( \frac{1}{{n}_{i}^{′}} - \frac{1}{n} ) ,$$
rᵢ denotes a radius of curvature of an i^{th} transmitting surface,
n′ᵢ denotes a refractive index at an emergence side of the i^{th} transmitting surface,
nᵢ denotes a refractive index at an incidence side of the i^{th} transmitting surface,
n′ denotes a refractive index of an image space,
i denotes a number of a transmitting surface, and
k denotes the total number of transmitting surfaces.

2. The endoscope according to claim 1, wherein the following conditional expression (2) is satisfied:
0.1 mm<f<0.8 mm (2),
where,
f denotes a focal length of the image forming optical system.

3. The endoscope according to claim 1, wherein the following conditional expression (3) is satisfied:
θout<θin (3),
where,
θin is an angle made by a principal light ray and a normal of the inner peripheral surface, in a first space (however, θ≠0),
θout is an angle made by the principal light ray and a normal of the outer peripheral surface, in a second space,
the first space is a space between the image forming optical system and the inner peripheral surface,
the second space is a space at an outer side of the circular cylindrical member, and
the principal light ray is a principal light ray from an object point of a center when the circular cylindrical member is measured in an optical axial direction.

4. The endoscope according to claim 1, wherein the following conditional expression (4) is satisfied:
1<R2/R1<5 (4),
where,
R1 denotes a radius of curvature of the inner peripheral surface, and
R2 denotes a radius of curvature of the outer peripheral surface.

5. The endoscope according to claim 1, wherein the following conditional expression (5) is satisfied:
1≤OB/R2<10 (5),
where,
OB denotes a distance from the optical axis up to the object plane, in a plane orthogonal to the optical axis, and
R2 denotes a curvature of the outer peripheral surface.

6. The endoscope according to claim 1, wherein
the image forming optical system includes in order from the distal end member side, a first positive lens and a second positive lens,
a first predetermined surface is a lens surface on the image plane side of the first positive lens,
a second predetermined surface is a lens surface on the distal end member side of the second positive lens,
the first predetermined surface is a convex surface toward the second positive lens, and
the second predetermined surface is a convex surface toward the first positive lens.

7. The endoscope according to claim 6, wherein the following conditional expressions (6) and (7) are satisfied:
0.5<ϕ1/ϕ<5.0 (6), and
0.1<ϕ2/ϕ<2.0 (7),
where,
ϕ denotes a refractive power of the image forming optical system,
ϕ1 denotes a refractive power of the first predetermined surface, and
ϕ2 denotes a refractive power of the second predetermined surface.

8. The endoscope according to claim 1, wherein the image forming optical system includes a planoconvex lens.

9. The endoscope according to claim 1, wherein the image forming optical system includes a ball lens.

10. The endoscope according to claim 1, wherein the image forming optical system includes a gradient index lens.

11. The endoscope according to claim 1, wherein
the image forming optical system is disposed at a distal end of an insertion portion of the endoscope,
the distal end of the insertion portion has a connecting portion,
the circular cylindrical member has a connecting portion on the other end, and
the circular cylindrical member is attached to and detached from the insertion portion via the two connecting portions.

12. The endoscope according to claim 1, wherein
the image forming optical system is disposed at a distal end of an insertion portion of the endoscope, and
the circular cylindrical member is fixed to the distal end of the insertion portion all the time.
